(19) United States
(12) Patent Application Publication
(10) Pub. No.: US 2023/0137422 A1
(43) Pub. Date: May 4, 2023

(54) COMPOSITION HAVING EXCELLENT PHYSICAL PROPERTIES, BIOSTABILITY, AND EFFECTS OF REDUCING BACTERIAL ADHESION AND BIOFILM FORMATION AND OF AMPLIFYING SOD ACTIVITY, FOR ORTHODONTIC CORRECTION AND PREVENTION AND TREATMENT OF DENTAL DISEASES

(71) Applicants: TOTAL MEDICAL SOLUTIONS HEALTHCARE CO., LTD., Cheongju-si (KR); INDUSTRY-ACADEMIC COOPERATION FOUNDATION, YONSEI UNIVERSITY, Seoul (KR)

(72) Inventors: Byung Youn CHOI, Cheongju-si (KR); Sang Hoon LEE, Cheongju-si (KR); Mee Ae KIM, Cheongju-si (KR); Geun LEE, Cheongju-si (KR); Jae Sik KIM, Cheongju-si (KR); Sung Hwan CHOI, Seoul (KR); Jae Sung KWON, Seoul (KR); Jie JIN, Seoul (KR); Ji Young SEO, Seoul (KR)

(21) Appl. No.: 17/541,835

(22) Filed: Dec. 3, 2021

(30) Foreign Application Priority Data
Oct. 28, 2021 (KR) 10-2021-0146000

(51) Int. Cl. A61K 8/46; A61K 8/25; A61Q 11/00; A61K 8/19

(52) U.S. Cl. CPC A61K 8/466; A61K 8/25; A61Q 11/00; A61K 8/19

(57) ABSTRACT

Proposed is a composition for orthodontic correction and prevention and treatment of dental disorders, and the composition contains mesoporous silica particles and a zwitterionic material. The composition may reduce thickness and biomass of a saliva-derived biofilm, increase antifouling ability, and reduce viability of bacteria due to an adhesion resistance to fungi and bacteria. In addition, the composition may prevent protein adsorption, provide excellent wettability and mechanical properties, provide resistance to inflammation through amplification of SOD activity, and prevent aggregation of ceria particles. Due to the advantages stated above, a new dental material that overcomes the problems of existing dental materials may be provided.

### CROSS REFERENCE TO RELATED APPLICATION

The present application claims priority to Korean Patent Application No. 10-2021-0146000 filed on Oct. 28, 2021, which is incorporated herein by reference in its entirety.

### BACKGROUND

### 1. Technical Field

The present invention relates to a composition for orthodontic correction and prevention and treatment of dental diseases. More specifically, the present invention relates to a composition for orthodontic correction and prevention and treatment of dental diseases, the composition being obtained by chemically changing the physical properties of PMMA with the use of mesoporous silica particles and a zwitterionic material, thereby having excellent physical properties, stability, and the effects of reducing proliferation of bacteria and formation of biofilm and of amplifying SOD activity.

### 2. Description of the Related Art

The global market size of new biocompatible material-related medical devices in fields such as orthopedics, dentistry, and the like was expected to grow to USD 55.4 billion by 2020, and the market size in Korea was expected to expand to about KRW 2.72 trillion. Polymethyl methacrylate (PMMA) is a representative biomaterial used in the healthcare field, and the market for PMMA is currently growing at an annual growth rate of 8% to 9%. The global market for PMMA is expected to grow from USD 4.72 billion in 2018 to USD 7.6 billion by 2025. In the field of dentistry, the market for dentures made of PMMA is expanding globally because of an aging population. In Korea, health insurance coverage of dentures for the elderly has been provided for those aged 75 and older since 2012 and expanded to include those aged 65 and older since 2016.

In addition, the orthodontic market for improving facial esthetics is growing rapidly due to a contemporary trend of putting emphasis on improving the quality of life regardless of one's age, and most removable orthodontic appliances are made of PMMA. The global orthodontic market is expected to grow from USD 4.32 billion in 2018 to USD 6.63 billion by 2023 at an annual growth rate of 8.9%. Considering this trend, source technology development for premium PMMA to be used for treatment is expected to lead to significant market expansion and creation of growth opportunities.

However, most of the PMMA used in dentistry is entirely dependent on imports from Ivoclar Vivadent Inc. located in the Principality of Liechtenstein and Lang Dental Manufacturing Company, Inc. located in USA. There are on-going research and development activities for antibacterial biomaterials in Korea and abroad to secure global competitiveness, but product development has not been successful due to several limitations. In particular, there are no PMMA products for treatment having an effect of inhibiting adhesion of biofilms or oral bacteria.

Oral biofilm has the most complex structure among biofilms in the human body since oral biofilm is formed in the oral cavity, where more than 700 types of various microorganisms reside, and oral biofilm is a cause of oral diseases and related systemic diseases. Oral biofilm is also pointed out as a cause of salivary proteins and local diseases in the oral cavity such as dental caries, periodontitis, apical inflammation, and peri-implantitis, as well as infectious systemic diseases affecting the digestive system and cardiovascular system.

Meanwhile, multi-drug resistance of bacteria in biofilms due to the indiscriminate use of antibacterial agents or antibiotics has been reported. A large amount of antibacterial agents can exhibit toxicity, which destroys normal cells. Therefore, use of a large amount of antibacterial agents is not in accordance with ISO standards and has a high probability of being toxic. In addition, when applied to a previously developed dental material or composition, a large amount of antibacterial agents deteriorates and changes mechanical and chemical properties of the dental material or composition so that the dental material or composition exhibits properties which are mechanically or chemically unsuitable. Because dentures, removable orthodontic appliances, and the like are worn in the oral cavity for a very long time, bacterial growth and tartar formation frequently occur due to biofilm adhesion. A simple mechanical removal of the biofilm causes dilution of an antibacterial agent used and may cause recurrent infectious disease due to damage or infection of the surrounding tissue during the removal process. In addition, when a cleaning method using a brush or the like is used, the surface of the device may be damaged, and the lifespan of the device may also be reduced.

Therefore, the present invention is intended to provide a new technology for stable application of PMMA in a complex and dynamic oral environment without causing multi-drug resistance of oral bacteria and toxicity to surrounding normal tissue.

Korean Patent No. 10-219837 (Dec. 28, 2020) describes a dental composition containing detonation nanodiamond (DND) and polymethyl methacrylate (PMMA) and a method of manufacturing an orthodontic device containing the dental composition.

Korean Patent No. 10-2114824 (May 19, 2020) describes a dental varnish containing a zwitterionic material and a fluoride-releasing material as active ingredients and a composition for prevention and treatment of dental caries containing the dental varnish.

### SUMMARY

An objective of the present invention is to provide a dental composition which is obtained by chemically changing the physical properties of PMMA using mesoporous silica particles and a zwitterionic material. The composition has an adhesion resistance to saliva-derived biofilm, fungi, and bacteria, an effect of preventing protein adsorption, an antifouling ability, and an effect of amplifying SOD activity. In addition, the composition is confirmed to be stable and multi-functional while maintaining the original wettability and mechanical properties of PMMA.

Another objective of the present invention is to provide a dental composition that addresses the problems with ceria such as deterioration of physical properties, cytotoxicity, and self-aggregation.

The present invention provides an orthodontic composition containing mesoporous silica and zwitterions.

In the orthodontic composition according to the present invention, the mesoporous silica may be preferably in a state in which CeO₂ is inserted into the pores of the mesoporous silica and bonded to the silica.

In addition, in the orthodontic composition of the present invention, the zwitterion may be preferably sulfobetaine methacrylate (SBMA).

The present invention provides a composition for prevention and treatment of dental diseases containing mesoporous silica and zwitterions.

In the composition for prevention and treatment of dental diseases according to the present invention, the mesoporous silica is preferably in a state in which CeO₂ is inserted into the pores of the mesoporous silica and bonded to the silica.

In addition, in the composition for prevention and treatment of dental diseases according to the present invention, the zwitterion is preferably SMBA.

The present invention provides a dental composition containing mesoporous silica particles and a zwitterionic material. The dental composition may reduce the thickness and biomass of a saliva-derived biofilm, increase antifouling ability against and reduce viability of fungi and bacteria as a result of adhesion resistance to fungi and bacteria, prevent protein adsorption, provide excellent wettability and mechanical properties, provide resistance to inflammation through amplification of SOD activity, and prevent aggregation of ceria particles. Due to the advantages stated above, a new dental material that overcomes the problems of existing dental materials may be provided.

### BRIEF DESCRIPTION OF THE DRAWINGS

The above and other objectives, features, and other advantages of the present invention will be more clearly understood from the following detailed description taken in conjunction with the accompanying drawings, in which:

FIG. 1 is a set of graphs showing results of mechanical testing using an SCZ (SBA-15-ceria (CeO₂), zwitterion) composition according to the present invention, in which FIG. 1 shows flexural strength (***P<0.001);

FIG. 2 is a set of graphs showing results of mechanical testing using an SCZ (SBA-15-ceria (CeO₂), zwitterion) composition according to the present invention, in which FIG. 2 shows elastic modulus (***P<0.001);

FIG. 3 is a set of graphs showing results of mechanical testing using an SCZ (SBA-15-ceria (CeO₂), zwitterion) composition according to the present invention, in which FIG. 3 shows Vickers hardness results (***P<0.001);

FIG. 4 is a graph showing results of a wettability test using the SCZ composition according to the present invention;

FIG. 5 is a graph showing the results of a cytotoxicity test using the SCZ composition according to the present invention (***P<0.001);

FIG. 6 is a graph showing results of a protein adsorption test using the SCZ composition according to the present invention (***P<0.001);

FIG. 7 show results of fungal and bacterial adhesion and viability tests using the SCZ composition according to the present invention, in which FIG. 7 shows representative images of stained live/dead Candida albicans and Streptococcus mutans attached to a sample surface (***P<0.001);

FIG. 8 show results of fungal and bacterial adhesion and viability tests using the SCZ composition according to the present invention, in which FIG. 8 respectively show WST coefficients derived from C. albicans attached to a surface of a material (***P<0.001);

FIG. 9 show results of fungal and bacterial adhesion and viability tests using the SCZ composition according to the present invention, in which FIG. 9 respectively show WST coefficients derived from S. mutans attached to a surface of a material (***P<0.001);

FIG. 10 show results of a test measuring a saliva-derived biofilm model and biomass using the SCZ composition according to the present invention, in which FIG. 10 shows representative images of stained live/dead biofilm attached to a sample surface (**P<0.01, ***P<0.001);

FIG. 11 show results of a test measuring a saliva-derived biofilm model and biomass using the SCZ composition according to the present invention, in which FIG. 11 shows quantitative analysis of biofilm thickness (**P<0.01, ***P<0.001);

FIG. 12 show results of a test measuring a saliva-derived biofilm model and biomass using the SCZ composition according to the present invention, in which FIG. 12 shows quantitative analysis of biofilm biomass (**P<0.01, ***P<0.001);

FIG. 13 is a graph showing the results of measuring SOD activity of the SCZ composition according to the present invention (***P<0.001);

FIG. 14 is graphs respectively showing XRD pattern of the SCZ composition according to the present invention, in which particle surface was analyzed using zeta potential.

FIG. 15 is graphs respectively showing surface charge of the SCZ composition according to the present invention, in which particle surface was analyzed using zeta potential.

### DETAILED DESCRIPTION

In the field of dentistry, use of PMMA having excellent physical properties is not limited to use in dental materials. However, in terms of therapeutic materials, there is no therapeutic PMMA product having an effect of inhibiting adhesion of biofilm and oral bacteria. Use of existing commercial PMMA is highly invasive. Therefore, the PMMA often became deteriorated and contaminated through an invasive interaction in the oral cavity, and the contaminated PMMA has caused various biofilms, tartar, bacterial growth, inflammation, and oral diseases. There have been studies to add various substances or compounds containing antibacterial and antifouling components to PMMA. Even when the PMMA with added substances or compounds exhibited efficacy, actual use proved difficult due to high cytotoxicity, non-conformance with ISO standards, and a reduction in mechanical/chemical properties and wettability.

Accordingly, in the present invention, a dental composition that conforms to ISO standards or exhibits excellent physical properties while reducing cytotoxicity, having the same mechanical strength as an existing PMMA composition, and amplifying SOD activity has been developed. In addition, the use of zwitterions made the following effects possible: antifouling ability against and reduced viability of fungi and bacteria, prevention of protein adhesion, and reduced biofilm thickness and biomass.

The present invention provides an orthodontic composition containing mesoporous silica and zwitterions. In addition, the present invention provides a composition for prevention and treatment of dental disorders containing mesoporous silica and zwitterions.

Meanwhile, in the composition according to the present invention, the mesoporous silica is a particle having mesopores. There are no limitations on the mesoporous silica as long as the mesoporous silica is known in the art. However, the mesoporous silica preferably has a molecular weight (m/w) of 30.0-500.0 and is SBA-15 having pores 4.0-30.0 nm in size and in a hexagonal arrangement. More preferably, the SBA-15 is in a state in which CeO₂ is inserted into the pores of the mesoporous silica and bonded to the silica (also referred to as “nanoceria (CeO₂)-modified SBA-15”). Nanoceria (CeO₂)-modified SBA-15 is prepared as described below. It is preferable to impregnate 0.5-1.0 parts by weight of SBA-15 and 1.2-1.4 parts by weight of Ce(NO₃)_{3*6}H₂O with aqueous citric acid solution, which was composed of 1.00-1.50 parts by weight of citric acid dissolved in 2-3 parts by weight of water, for 10-14 hours, followed by drying at 100° C.-140° C. and calcining at 300° C.-500° C. for 2-6 hours. Through this, nanoceria (CeO₂)-modified SBA-15 having a nominal loading of 20-60 wt % of CeO₂ may be obtained.

A zwitterion refers to an ion that has both positive and negative charges, and an amino acid includes an amino group (—NH₂) and a carboxyl group (—COOH). In the present invention, the zwitterion is preferably SBMA.

The composition according to the present invention preferably contains 1.5% (w/v) of SBA-15-ceria (CeO₂), 1.5% (w/v) of SBMA, 58.2% (w/v) of PMMA powder, and 38.8% (w/v) of PMMA liquid. More preferably, 1.5% (w/v) of nanoceria (CeO₂)-modified SBA-15 and 1.5% (w/v) of SBMA may be added to PMMA liquid and mixed through 1 hour of sonication and 4 hours of stirring. Then, the mixture may be mixed with PMMA powder at room temperature and polymerized for 30 minutes at 60° C. and an atmospheric pressure of 4.0 bar. There are no limitations on the mixing conditions as long as the mixing conditions are known in the art. However, mixing is preferably performed in a vacuum state or a nitrogen environment.

Based on the results of the following experiment, the composition according to the present invention is stable. The composition shows a value higher than the ISO specification in mechanical testing, has hydrophilicity that is equal to or improved compared to the hydrophilicity of an ordinary PMMA-based dental material in the wettability test, and has a cytotoxicity value that conforms to the ISO standard. In addition, the composition provides inflammation resistance and prevents aggregation of ceria particles by amplifying SOD activity, has the ability to prevent protein adsorption, has antifouling ability against and reduces viability of fungi and bacteria, and has an effect of reducing biofilm thickness and biomass. Accordingly, the composition may replace an existing PMMA composition and be used as a material for a new dental composition.

Meanwhile, the term “biofilm” used in the present invention is defined as a cluster of proteins, fungi, bacteria, and the like formed on the surfaces of teeth and therapeutic orthodontic appliances. A biofilm continues to increase in size due to the temperature and humidity in the oral cavity and food residues that nourish bacteria. A biofilm gives rise to cavities and inflammation of the gums in the oral cavity and causes gingivitis and periodontitis. Debris and proteins accumulated on the biofilm form plaque, which in turn becomes tartar. In order to block formation of plaque and tartar and promote oral health, resistance to adhesion of biofilm and protein and antifouling ability against bacteria and fungi are essential.

The term “biomass” used in the present invention may refer to the entire bacterial biomass of a cluster formed as a biofilm.

The composition according to the present invention may additionally contain at least one selected from the group consisting of a curing agent, a stabilizer, a pH adjuster, a flame retardant, a lubricant, a filler, an indentation-reducing agent, a fluorescent whitening agent, a catalyst for polycondensation, an antistatic agent, an antifoaming agent, an emulsifier, a thickener, a fragrance, and a reinforcing material. However, the additive is not limited to the above, and the composition may further include a variety of additives.

Hereinafter, the present invention will be described in more detail through the examples and experimental examples below.

However, the scope of the present invention is not limited to the examples and experimental examples below and also includes modifications equivalent to the technical spirit of the examples and experimental examples.

Example: Preparation of SCZ (SBA-15-Ceria (CeO₂), Zwitterion) Composition

In this example, an “SCZ (SBA-15-ceria (CeO₂), zwitterion) composition” was prepared. The SCZ composition is a composition containing SBA-15-ceria (CeO₂), which is SBA-15 modified with nanoceria (CeO₂), and zwitterions.

An incipient wetness impregnation technique with aqueous cerium(III) nitrate was applied to the preparation. To impregnate 0.8 g of SBA-15 (available from ACS Material, LLC located in Pasadena, Calif., USA), 1.35 g of Ce(No₃)_{3*6}H₂O and 1.19 g of citric acid were dissolved in 2.4 ml of water and the SBA-15 was added to the solution, followed by 12 hours of stirring. The SBA-15 used had a particle size of 1-4 μm, pores 6-11 nm in size and in a hexagonal arrangement, and a molecular weight (m/w) of 60.08. After the impregnation as described above, the sample was dried at 120° C. for 12 hours and calcined at 400° C. for 4 hours. Finally, the synthesis of ceria-modified SBA-15 (also referred to as “SBA-15-ceria (CeO₂)”) having a nominal loading of 40 wt % CeO₂ was completed. The CeO₂ was converted from aqueous cerium(III) nitrate through the following chemical reaction in the pores of SBA-15: Ce(No₃)_{3*6}H₂O=Heating=Ce(No₃)₃+6H₂O, Ce(No₃)₃=High temperature=CeO₂+3NO⬆+2O₂⬆.

1.5%-3% of SBA-15-ceria (CeO₂) prepared above and 1.5%-3% of SBMA were added to PMMA liquid and mixed through 1 hour of sonication and 4 hours of stirring. The mixed liquid and PMMA powder were mixed at room temperature to complete the SCZ composition. Then, defoaming and molding according to the purpose were performed at 60° C. in a pressurized environment (polymerization at 4.0 bar for 30 minutes).

In order to evaluate the physical properties and effectiveness of the SCZ composition according to the present invention, compositions including a control group and experimental groups for comparison were prepared as shown in Table 1 and used in the experiments described below.

TABLE 1

| | | Ceria | SBA-15-Ceria | Zwitterion | PMMA^{b} |
|---|---|---|---|---|---|
| Groups | SBA-15 | (CeO₂) | (CeO₂) | (SBMA^{a}) | powder/liquid |
| Control^{c} | — | — | — | — | 60/40 |
| S | 1.5 | — | — | — | 59.1/39.4 |
| C | — | 1.5 | — | — | 59.1/39.4 |
| SC1 | — | — | 1.5 | — | 59.1/39.4 |
| SC1Z1 | — | — | 1.5 | 1.5 | 58.2/38.8 |
| SC1Z2 | — | — | 1.5 | 3 | 57.3/38.2 |
| SC2 | — | — | 3 | — | 58.2/38.8 |
| SC2Z1 | — | — | 3 | 1.5 | 57.3/38.2 |
| SC2Z2 | — | — | 3 | 3 | 56.4/37.6 |

^{a}sulfobetaine methacrylate (SBMA),
^{b}polymethyl methacrylate (PMMA) (Physical properties cannot be guaranteed when the SBA-15-ceria content exceeds 5%)
^{c}The control refers to properties of ordinary commercial PMMA alone.

Experimental Example 1: Evaluation of Mechanical Properties, Structural Stability, Wettability, Cytotoxicity, and Conformity to ISO Standards for SCZ (SBA-15-Ceria (CeO₂), Zwitterion) Composition of Example

In this experimental example, mechanical properties, structural stability, wettability, and cytotoxicity of the SCZ (SBA-15-ceria (CeO₂), zwitterion) composition were evaluated, and conformity to ISO standards was checked on the basis of the evaluation results.

1) Evaluation of Mechanical Properties and Structural Stability

Mechanical properties were evaluated according to ISO 20795-2[3]. A sample was prepared with dimensions of 3.3 mm (height)×10 mm (width)×64 mm (length). A three-point bend test was performed using a universal tester by model name of Instron 3366 available from Instron. Flexural strength and modulus of elasticity were measured at a span length of 50 mm and a crosshead speed of 5 mm/min. Flexural strength and modulus of elasticity were calculated according to the standard formula defined by ISO. Vickers hardness was measured for 30 seconds under a test load of 300 gf (2.94 N) using a hardness tester by model name of DMH-2 available from Matsuzawa Seiki Co., Ltd. An average value for each sample was calculated from measurements at three locations.

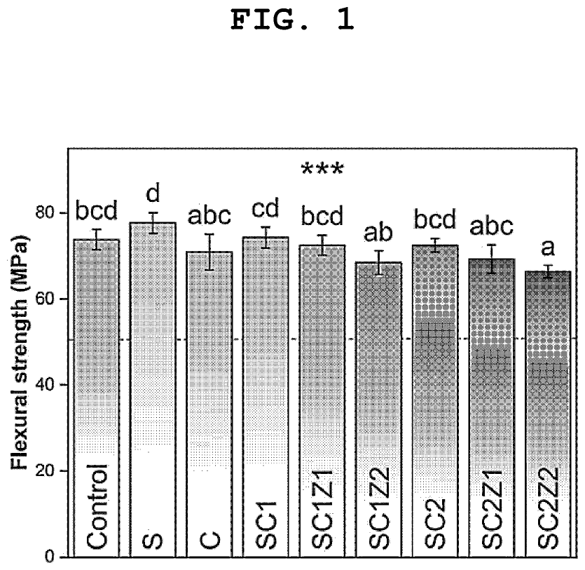

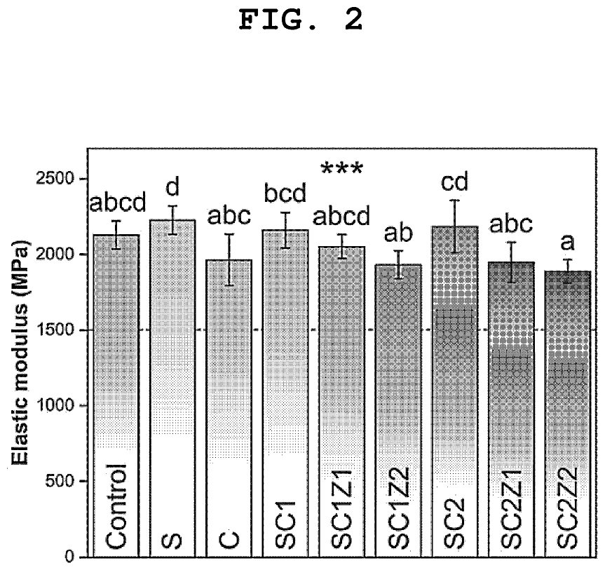

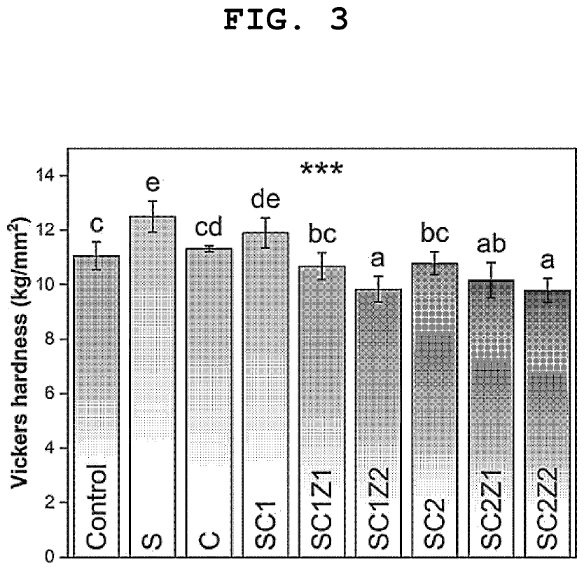

The mechanical property results were as shown in FIGS. 1 to 3. When SBA-15 (represented by “S”) was added, the mechanical properties of PMMA were greatly improved. On the other hand, it was found that an increase in the contents of ceria and zwitterions had a negative impact on the mechanical properties. In all of the groups (even SC2Z2, which showed the lowest mechanical properties), the flexural strength, modulus of elasticity, and Vickers hardness tendencies were excellent and showed values higher than ISO specifications (represented by blue dotted lines on the graph). This is thought to be attributable to the characteristics of SBA-15.

2) Verification of Wettability

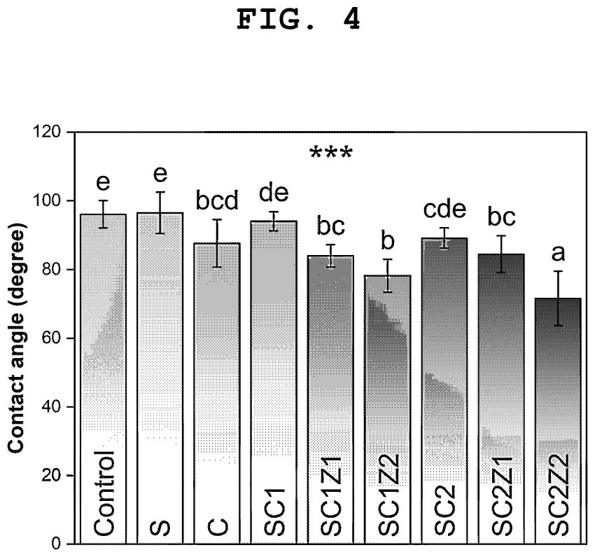

It was intended to check whether the composition according to the present invention had a satisfactory physical property compared to a composition that is already in use. Disk-shaped samples (15 mm in diameter and 2 mm in thickness) were prepared using a standardized polyacetal resin mold. 5 pL of distilled water (DW) was dropped on a surface of a sample under dry conditions. After 10 seconds, contact angle was measured using a video contact angle goniometer by product name of SmartDrop available from Femtobiomed Inc. For each sample, two measurements were taken, and the average was recorded. As a result of measuring the contact angle, as shown in FIG. 4, the control, S, SC1, and SC2 groups showed a slight downward trend, but the difference was not large. This indicates that SBA-15 had little effect on the hydrophilicity of PMMA. It can be seen that the contact angle of the Ceria group decreased. This indicates that the hydrophilicity of PMMA improved. The contact angle of the groups to which zwitterions were added (SC1Z1, SC1Z2, SC2Z1, and SC2Z2) was significantly reduced, which was consistent with previous studies. Through this, it was confirmed that the composition according to the present invention has hydrophilicity that is similar to or improved compared to an ordinary PMMA-based dental material.

3) Verification of Cytotoxicity

It was intended to check the presence of cytotoxicity of the composition according to the present invention and at the same time, check conformity of the cytotoxicity value to an ISO standard. Cytotoxicity was evaluated through MTT analysis, and MTT analysis was performed according to ISO 10993-5[4]. L929 cells were inoculated into a 96-well plate at 1×10⁵ cells/mL and then incubated at 37° C. for 24 hours. Sampling was prepared according to ISO 10993-12[5]. After a semi-fused monolayer was formed in the wells, the culture medium was removed from the wells, and 100% extract having a volume of 100 mL was added to each well. A positive control was a 1% phenol solution, and a negative control was an extract of aluminum oxide ceramic. After 24 hours, the medium was replaced with 50 mL of thiazolyl blue tetrazolium bromide (MTT) solution, which is available from Sigma-Aldrich Inc. After replacing the MTT solution with 100 mL of isopropanol, the plate was gently shaken to dissolve the crystals. Absorbance at 570 nm was measured using a microplate reader by product name of Epoch available from BioTek Instruments.

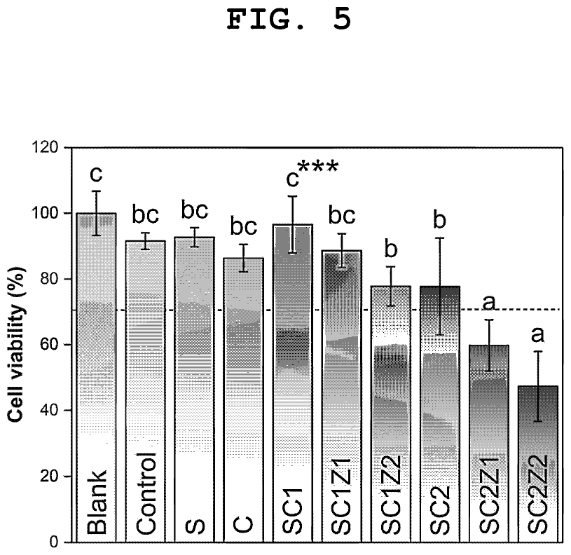

As a result, as shown in FIG. 5, the control, S, C, SC1, and SC1Z1 groups had low cytotoxicity, and among the groups containing zwitterions, the SC1Z1 group showed the highest cell viability. In addition, the cell viability of SC1Z2 and SC2 was close to the ISO standard (represented by a blue dotted line on the graph) but showed a significant decrease compared to the control. The cell viability of SC2Z1 and SC2Z2 was lower than the ISO standard, indicating potential cytotoxicity. Therefore, the composition according to the present invention was found to be safe for use when used within a range that does not exhibit cytotoxicity.

Experimental Example 2: Verification of Effectiveness of Prevention of Protein Adsorption by SCZ (SBA-15-Ceria (CeO₂), Zwitterion) Composition of Example

In this experimental example, it was intended to verify effectiveness of prevention of protein adsorption by the SCZ (SBA-15-ceria (CeO₂), zwitterion) composition.

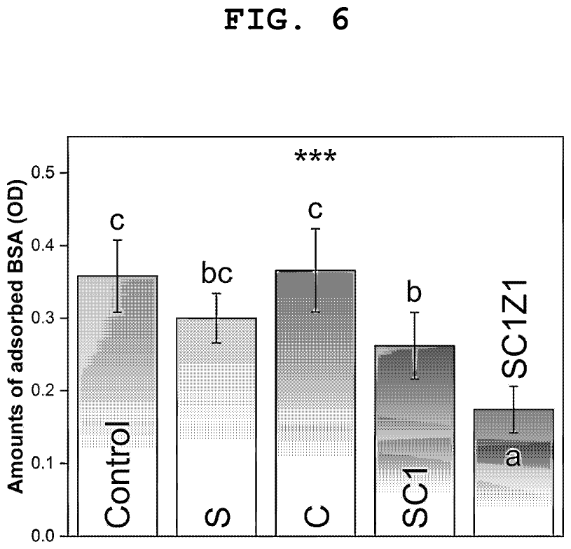

For evaluation of initial protein adsorption onto a sample surface, disk-shaped samples were prepared (15 mm in diameter and 2 mm in thickness) and immersed in fresh phosphate buffered saline (PBS), which is available from Gibco, at room temperature for 1 hour. Each sample was then immersed in a bovine serum albumin (BSA, available from Pierce Biotechnology Inc.) broth (100 pL of PBS containing 2 mg of protein/mL). After incubation at 37° C. in 5% CO₂ for 4 hours, proteins not attached to the sample were removed by washing twice with PBS. Then, the amount of protein attached to the sample was measured using micro-bicinchoninic acid (200 pL from Micro BCA™ Protein Assay Kit available from Pierce Biotechnology Inc.), and incubation was performed at 37° C. for 30 minutes. The amount of protein adsorbed onto the surface was quantified on the basis of optical density (OD) at 562 nm measured using a microplate reader by product name of Epoch available from BioTek Instruments. As a result, as shown in FIG. 6, the amount of adsorbed BSA was significantly lower in the SC1Z1 group than in the other groups. Such a result shows zwitterions' characteristic of repelling protein adsorption, confirming that the composition according to the present invention had an excellent effect of preventing protein adsorption.

Experimental Example 3: Confirmation of Resistance to Adhesion of Fungi and Bacteria of SCZ (SBA-15-Ceria (CeO₂), Zwitterion) Composition of Example and Reduction in Viability of Fungi and Bacteria due to Composition

In this experimental example, it was intended to check resistance to adhesion of fungi and bacteria of the SCZ (SBA-15-ceria (CeO₂), zwitterion) composition) and a reduction in viability of fungi and bacteria due to the composition.

Microbial analysis was performed according to a previously established method. Disk-shaped samples were prepared (10 mm in diameter and 2 mm in thickness). Fungal and bacterial analyses were performed using Candida albicans (Korean Collection for Oral Microbiology (KCOM) 1301) and Streptococcus mutans (ATCC 25175). Using a 24-well plate, a fungal or bacterial suspension (1 mL, 1×10⁸ cells/mL) was added to each sample and then incubated at 37° C. for 24 hours. After incubation, fungi or bacteria not attached to the sample were removed by washing twice with PBS. Bacteria attached to a surface of the sample were collected by ultrasonication (using SH-2100, available from Saehan Ultrasonic) in 1 mL of Brain Heart Infusion for 5 minutes.

Microbial Viability Assay Kit-WST (available from Dojindo Molecular Technologies Inc. headquartered in Kumamoto, Japan) was used according to the manufacturer's technical manual as a colorimetric indicator whose results are directly proportional to the number of living microorganisms. Using a 96-well plate, 10 μl of a staining reagent was added to the harvested bacterial suspension (190 μl), incubation was performed at 37° C. for 2 hours, and then absorbance at 450 nm was measured using a microplate reader by product name of Epoch available from BioTek Instruments. An average of 3 experiments was determined as the result.

A Live/Dead bacterial viability kit, which is available from Molecular Probes headquartered in Eugene, Oreg., USA, was used to examine the viability of attached bacteria according to the manufacturer's protocol. C. albicans and S. mutans were cultured in the same manner as described above. Stained samples were observed under a confocal laser scanning microscope (CLSM) by product name of LSM880 available from Carl Zeiss, Inc. headquartered Thornwood, N.Y., USA. Live bacteria were shown in green and dead bacteria were shown in red.

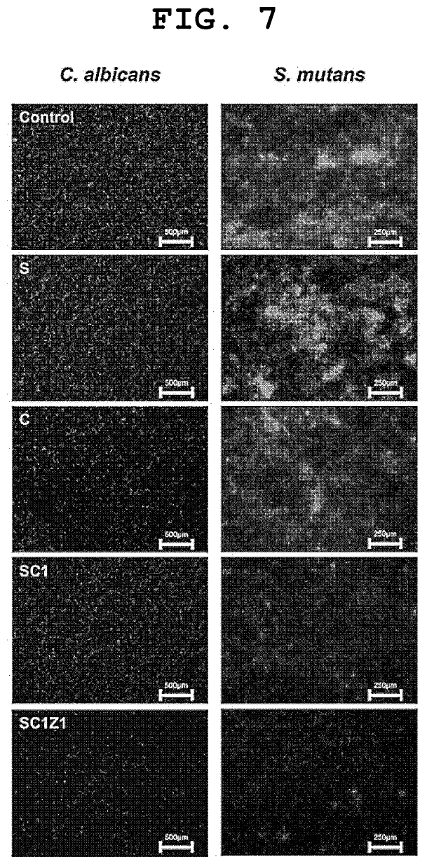

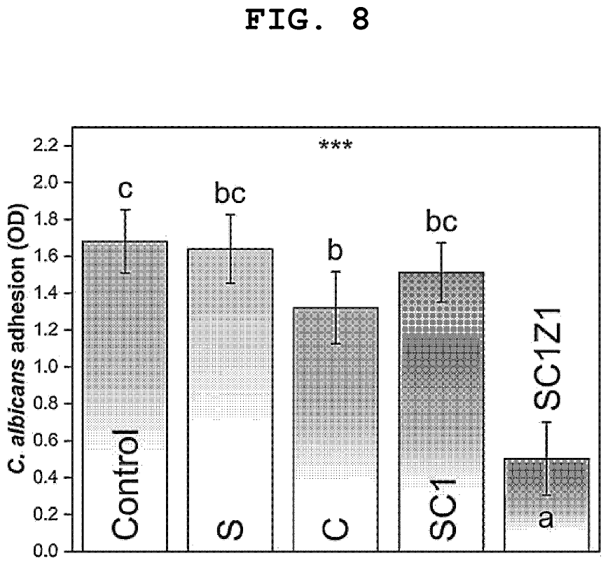

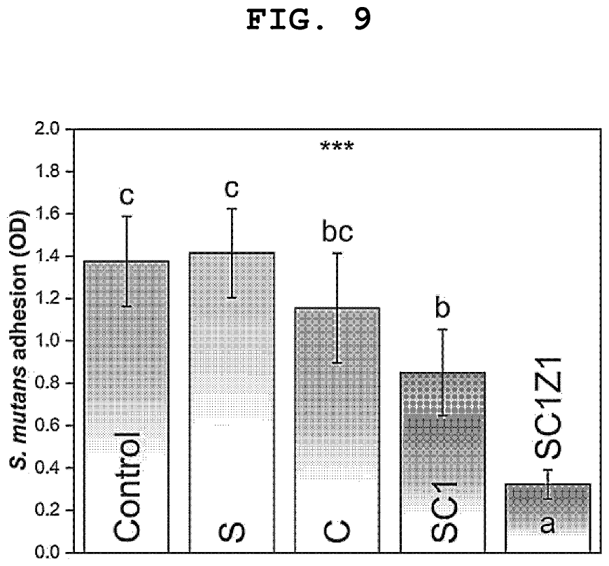

As a result, as shown in FIGS. 7 to 9, all groups were shown to be covered with live bacteria (stained in green), and the control group and the S group showed comparable levels of adhesion, which were higher than those of the other groups. In the C. albicans environment, the adhesion of the C group was lower than that of the SC1 group. This is due to a tendency of C. albicans having better adhesion to a hydrophobic surface. In the S. mutans environment, the adhesion of the SC1 group was lower than that of the C group. This is due to SBA-15-ceria having a greater antibacterial effect than ceria. In particular, SC1Z1 showed the lowest levels of adhesion among all of the groups in both the fungal (C. albicans) and bacterial (S. mutans) environments. Through this, it was confirmed that the composition according to the present invention has excellent resistance to fungal and bacterial adhesion and therefore shows an outstanding antifouling ability against fungi and bacteria and an outstanding effect of reducing the viability of fungi and bacteria.

Experimental Example 4: Confirmation of Antifouling Effectiveness of SCZ (SBA-15-Ceria (CeO₂), Zwitterion) Composition of Example against Saliva-derived Biofilm Model and Biomass

In this example, it was intended to check antifouling effectiveness of the SCZ (SBA-15-ceria (CeO₂), zwitterion) composition against a saliva-derived biofilm model and biomass.

Measurements of saliva-derived biofilm model and biomass and analysis of human saliva-derived biofilm were performed according to previously established methods. Saliva was collected according to the procedures approved by the Institutional Review Board (2-2019-0049) of Yonsei University Dental Hospital located in Seoul, Korea in accordance with the ethical principles of the Declaration of Helsinki adopted at the 64th General Assembly of the World Medical Association. Written consent was obtained from all participants prior to saliva donation. Human saliva from 6 adults was mixed in equal proportions, diluted to 30% in sterile glycerol, and stored at −80° C. A biofilm model was cultured in McBain medium to simulate a salivary environment and secure an environment for stable microbial growth. 1.5 mL of the culture fluid was dropped on each sample (10 mm in diameter and 2 mm in thickness), and the biofilm was incubated at 37° C. for 48 hours. Additional 1.5 mL of culture medium was added after 8, 16, and 24 hours. Samples were stained with a Live/Dead bacterial viability kit, which is available from Molecular Probes headquartered in Eugene, Oreg., USA, according to the manufacturer's protocol. Five sites were randomly selected under CLSM to observe the biofilm on the surface of the sample. Biofilm thickness was measured using ZEN software from Carl Zeiss, Inc. with respect to the vertical axis of the image. Average biomass was measured using ImageJ software from NIH with COMSTAT plugin from Danish Technical University.

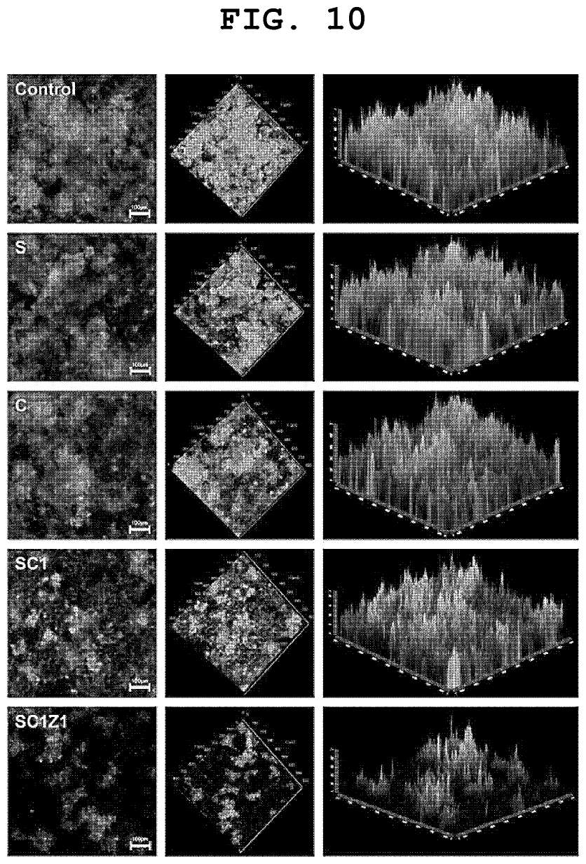

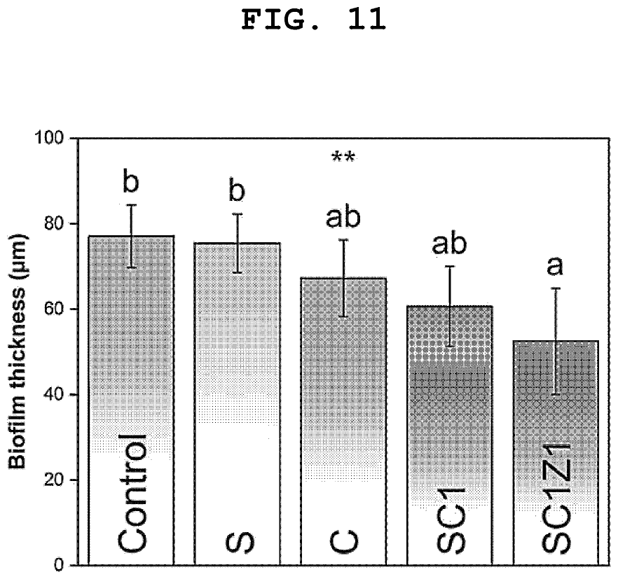

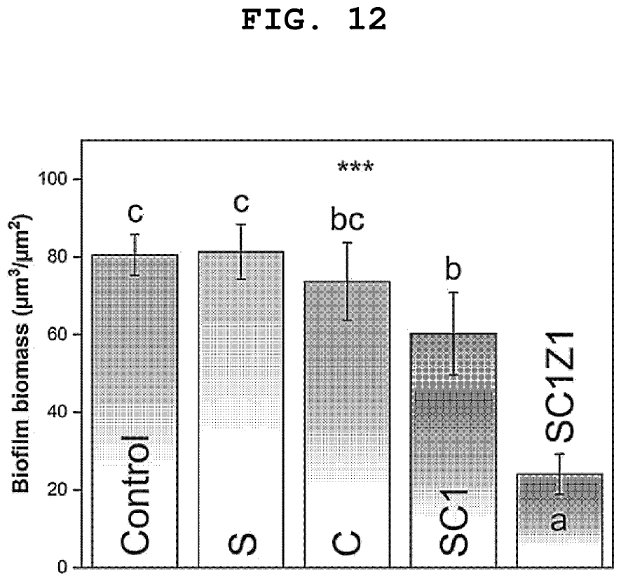

As a result, FIGS. 10 to 12 show biofilm thickness and biomass for different groups obtained for a single type of bacteria. Biofilm biomass and thickness were significantly decreased in the SC1 and SC1Z1 groups compared to the control and S groups. The SC1 sample did not differ significantly from the control sample in terms of biofilm thickness, but biofilm biomass of the SC1 sample was significantly smaller than that of the control. In particular, the SC1Z1 group had the smallest amount of biofilm formation while also having a small biofilm thickness. Through this, it was confirmed that the composition according to the present invention exhibits antifouling effectiveness against biofilm and biomass.

Experimental Example 5: SOD Mimetic Activity Assay of SCZ (SBA-15-Ceria (CeO₂), Zwitterion) Composition of Example

In this experimental example, it was intended to perform SOD mimetic activity assay on the SCZ (SBA-15-ceria (CeO₂), zwitterion) composition.

SOD mimetic activity assay was performed after sampling prepared according to ISO 10993-12. A group's SOD activity was measured with Superoxide Dismutase Assay Kit, which is available from DoGenBio Co., Ltd. located in Seoul, Korea, according to the manufacturer's instructions. To give a brief description, WST-8 can react with a superoxide radical anion (.O₂⁻) generated by xanthine oxidase and produce a water-soluble formazan dye. The reaction may be blocked by SOD because SOD can catalyze dismutation of .O₂⁻ to produce hydrogen peroxide (H₂O₂) and O₂. Therefore, the production of formazan dye has a negative correlation with SOD activity. Absorbance at 450 nm was measured after incubation. Inhibition rate (%) represents the SOD activity of each group.

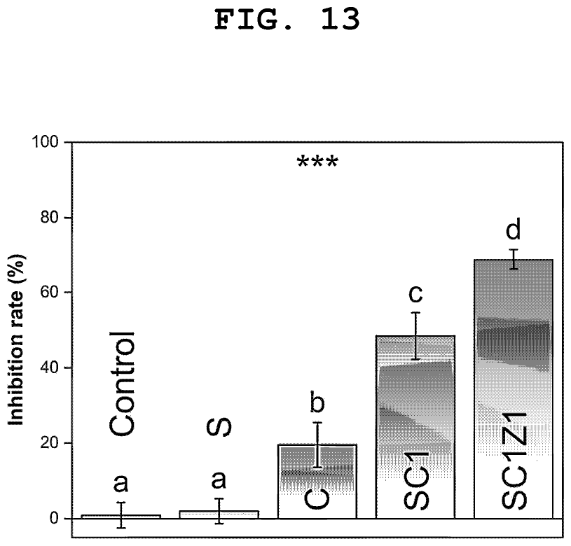

In a cellular environment, .O₂⁻ serves as a signaling molecule and is produced as a result of normal cellular metabolism. However, background .O₂⁻ levels can rise rapidly during an inflammatory response. SOD activity represents an ability to scavenge .O₂⁻ and may be considered as a potential to resist inflammation. As shown in FIG. 13, the control group and S group did not show SOD activity. However, the SC1 and SC1Z1 groups showed SOD activities which were higher than that of the C group. In particular, the SC1Z1 group showed the highest SOD activity. It can be explained that while aggregation of nanoceria particles affects Ce3+-Ce4+ conversion and reduces SOD activity, SBA-15 helps dispersion of ceria and zwitterions help ions diffuse to the surface of a material, thereby increasing the SOD activity. As described above, the composition according to the present invention exhibits an ability to scavenge regenerated reactive oxygen species (O₂⁻) through excellent SOD activity. Accordingly, the composition can exhibit inflammatory resistance, thereby relieving or reducing inflammation, accelerating healing of wounds, and inhibiting bacterial growth.

Experimental Example 6: Confirmation of Prevention of Aggregation of Ceria (CeO₂) by SCZ (SBA-15-Ceria (CeO₂), Zwitterion) Composition of Example

In this experimental example, it was intended to check prevention of aggregation of ceria (CeO₂) by the SCZ (SBA-15-ceria (CeO₂), zwitterion) composition.

Commercially available SBA-15, nanoceria, and synthesized SBA-15-ceria were characterized. Phase composition of a material was analyzed by collecting powder X-ray diffraction (XRD) data using Miniflex 600 Diffractometer, available from Rigaku, with a monochromator and Cu Kα radiation (λ=1.5418 Å). Diffraction patterns were collected in a 2θ range of 2° to 90° at a scanning rate of 0.2 deg/min. Phase constitution analysis was performed using PCPDFWIN database and POWDER CELL 2.4, which is a full profile analysis program. Surface charges of SBA-15, nanoceria, and synthesized SBA-15-ceria were measured with a zeta potential analyzer by product name of Zetasizer Nano ZS available from Malvern Pananalytical Ltd. headquartered in the UK. 1 mg of material was dispersed in 1 mL of DW for 30-40 minutes using an ultrasonic machine. The well-dispersed ceria nanoparticles were further diluted 10-fold in DW for measurement of surface charge.

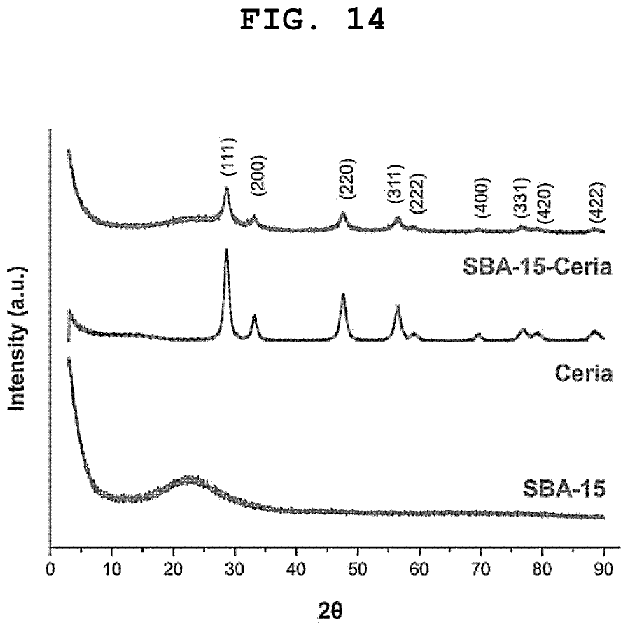

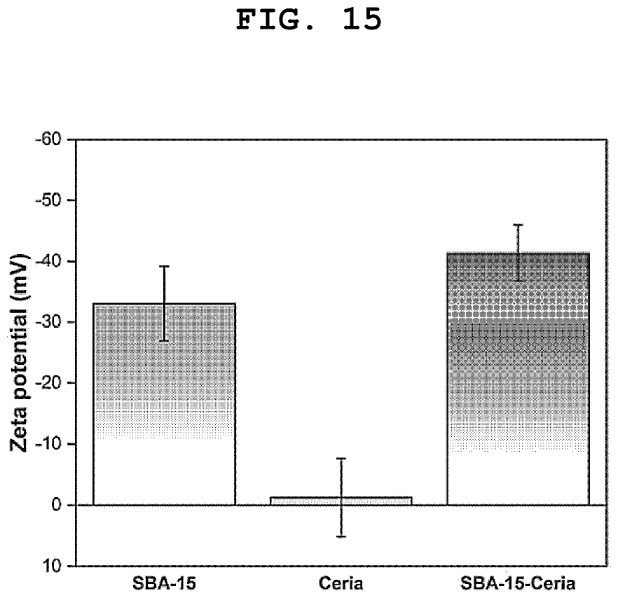

As a result, XRD patterns in a range of 2° to 90° for SBA-15, ceria and SBA-15-ceria are shown in FIG. 14. A peak at 23° of 2θ in the XRD patterns and reflection at an angle smaller than 5° are typical for SBA-15. Reflections of cerium oxide at 28.6°, 32.9°, 47.2°, 56.3°, 76.6°, and 88.3° of the 2θ region were observed in the SBA-15-ceria sample. Intensity of small angle reflection of SBA decreases as a result of addition of cerium oxide because the porous space of SBA-15 gets filled with ceria, which means a partial decrease in the regular porous structure of SBA-15. A surface charge of a particle was mainly determined by zeta potential analysis, and as shown in FIG. 15, conditions showing the surface charge of SBA-15, ceria, and SBA-15-ceria at pH 7 were −33.0±6.2 mV, −1.3±6.4 mV, and −41.3±4.57 mV, respectively. A high zeta potential value indicates a highly charged particle. The high charge prevents aggregation of particles through electric repulsion and leads to better dispersion. Aggregation is a major problem affecting functions of nanomaterials. The composition according to the present invention prevents aggregation of particles through electric repulsion, and when combined with SBA-15, ceria has better dispersion and activity.

What is claimed is:

1. An orthodontic composition containing mesoporous silica and zwitterions.

2. The orthodontic composition of claim 1, wherein the mesoporous silica is in a state in which CeO₂ is inserted into the pores of the mesoporous silica and bonded to the silica.

3. The orthodontic composition of claim 1, wherein the zwitterion is sulfobetaine methacrylate.

4. A dental composition for prevention and treatment of dental diseases, the composition containing mesoporous silica and zwitterions.

5. The dental composition of claim 4, wherein the mesoporous silica is in a state in which CeO₂ is inserted into the pores of the mesoporous silica and bonded to the silica.

6. The dental composition of claim 4, wherein the zwitterion is sulfobetaine methacrylate.
